(19) United States
(12) Patent Application Publication
(10) Pub. No.: US 2008/0288017 A1
(43) Pub. Date: Nov. 20, 2008

(54) External Baroreflex Activation

(75) Inventors: Robert S. Kieval, Medina, MN (US); Martin A. Rossing, Coon Rapids, CA (US); Robert J. Cody, Minneapolis, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, INC., Minneapolis, MN (US)

(21) Appl. No.: 12/038,707

(22) Filed: Feb. 27, 2008

Related U.S. Application Data
(60) Provisional application No. 60/883,721, filed on Feb. 27, 2007.

(51) Int. Cl. A61N 1/36

(52) U.S. Cl. 607/44

(57) ABSTRACT

Methods and systems for external baroreflex activation of a baroreceptor system of a patient from a stimulator external to the patient. The method and devices, enable baroflex therapy on a temporary basis and/or assess the response of a patient to such baroreflex therapy.

### CROSS REFERENCE TO RELATED APPLICATIONS

This application claims the benefit of U.S. Provisional Application No. 60/883,721 (Attorney Docket No. 021433-002500US), filed Feb. 27, 2007, the full disclosure of which is hereby incorporated by reference.

### BACKGROUND OF THE INVENTION

1. Field of Invention

The present invention generally relates to medical devices and methods for baroreflex activation. Specifically, the present invention relates to devices and methods for externally activating the baroreflex system on a temporary basis for medical conditions requiring temporary use of such methods and devices and/or for assessing the effect of such stimulation on the patient's baroreceptor system.

Cardiovascular disease is a major contributor to patient illness and mortality. It also is a primary driver of health care expenditure, costing more than $326 billion each year in the United States. Hypertension, or high blood pressure, is a major cardiovascular disorder that is estimated to affect over 60 million people in the United Sates alone. Of those with hypertension, it is reported that fewer than 30% have their blood pressure under control. Hypertension is a leading cause of heart failure and stroke. It is listed as a primary or contributing cause of death in over 200,000 patients per year in the U.S. Accordingly, hypertension is a serious health problem demanding significant research and development for the treatment thereof.

Hypertension occurs when the body's smaller blood vessels (arterioles) constrict, causing an increase in blood pressure. Because the blood vessels constrict, the heart must work harder to maintain blood flow at the higher pressures. Although the body may tolerate short periods of increased blood pressure, sustained hypertension may eventually result in damage to multiple body organs, including the kidneys, brain, eyes and other tissues, causing a variety of maladies associated therewith. The elevated blood pressure may also damage the lining of the blood vessels, accelerating the process of atherosclerosis and increasing the likelihood that a blood clot may develop. This could lead to a heart attack and/or stroke. Sustained high blood pressure may eventually result in an enlarged and damaged heart (hypertrophy), which may lead to heart failure.

Heart failure is the final common expression of a variety of cardiovascular disorders, including ischemic heart disease. It is characterized by an inability of the heart to pump enough blood to meet the body's needs and results in fatigue, reduced exercise capacity and poor survival. It is estimated that approximately 5,000,000 people in the United States suffer from heart failure, directly leading to 39,000 deaths per year and contributing to another 225,000 deaths per year. It is also estimated that greater than 400,000 new cases of heart failure are diagnosed each year. Heart failure accounts for over 900,000 hospital admissions annually, and is the most common discharge diagnosis in patients over the age of 65 years. It has been reported that the cost of treating heart failure in the United States exceeds $20 billion annually. Accordingly, heart failure is also a serious health problem demanding significant research and development for the treatment and/or management thereof.

Heart failure results in the activation of a number of body systems to compensate for the heart's inability to pump sufficient blood. Many of these responses are mediated by an increase in the level of activation of the sympathetic nervous system, as well as by activation of multiple other neurohormonal responses. Generally speaking, this sympathetic nervous system activation signals the heart to increase heart rate and force of contraction to increase the cardiac output; it signals the kidneys to expand the blood volume by retaining sodium and water; and it signals the arterioles to constrict to elevate the blood pressure. The cardiac, renal and vascular responses increase the workload of the heart, further accelerating myocardial damage and exacerbating the heart failure state. Accordingly, it is desirable to reduce the level of sympathetic nervous system activation in order to stop or at least minimize this vicious cycle and thereby treat or manage the heart failure.

A number of drug treatments have been proposed for the management of hypertension, heart failure and other cardiovascular disorders. These include vasodilators to reduce the blood pressure and ease the workload of the heart, diuretics to reduce fluid overload, inhibitors and blocking agents of the body's neurohormonal responses, and other medicaments.

Various surgical procedures have also been proposed for these maladies. For example, heart transplantation has been proposed for patients who suffer from severe, refractory heart failure. Alternatively, an implantable medical device such as a ventricular assist device (VAD) may be implanted in the chest to increase the pumping action of the heart. Alternatively, an intra-aortic balloon pump (IABP) may be used for maintaining heart function for short periods of time, but typically no longer than one month. Other surgical procedures are available as well.

2. Brief Description of the Background Art

It has been known for decades that the wall of the carotid sinus, a structure at the bifurcation of the common carotid arteries, contains stretch receptors (baroreceptors) that are sensitive to the blood pressure. These receptors send signals via the carotid sinus nerve to the brain, which in turn regulates the cardiovascular system to maintain normal blood pressure (the baroreflex), in part through modulation of the sympathetic and/or parasympathetic, collectively the autonomic, nervous system. Electrical stimulation of the carotid sinus nerve (baropacing) has previously been proposed for therapeutic purposes. For example, U.S. Pat. No. 6,073,048 to Kieval et al., the full disclosure of which is incorporated herein by reference, discloses a system and method for stimulating the carotid sinus nerve based on various cardiovascular and pulmonary parameters.

Devices and methods for externally stimulating baroreceptors to monitor and control a patient's blood pressure are described in U.S. Pat. Nos. 6,050,952 and 5,727,558 to Hakki et al., the full disclosures of which are incorporated fully herein by reference. These devices and methods, however, are designed only for therapeutic use and do not provide for external baroreflex activation to assess patient response, help a physician choose a location in the patient's body for placing the implant, or the like. Thus, currently available baroreflex activation treatments generally involve attaching cumbersome external devices to a patient or implanting an implantable device without knowing beforehand whether it will work for a given patient.

Therefore, a need exists for devices and methods for either or both providing temporary blood pressure control, and evaluating a patient's response to baroreflex activation before implanting an activation device in the patient. At least some of these objectives will be met by the present invention.

### BRIEF SUMMARY OF THE INVENTION

To address the problems of hypertension, heart failure, other cardiovascular disorders, nervous system and renal disorders, the present invention provides methods, devices (i.e., baroreflex activation device), and systems for practicing the same, by which at least one baroreflex system within a patient's body is activated by an external stimulus generator. In an embodiment, the activation by the external stimulus generator is on a temporary basis. When the baroreflex system is activated, the effects of such activation may include reducing excessive blood pressure, autonomic nervous system activity, and neurohormonal activation. Such activation systems suggest to the brain an increase in blood pressure and the brain in turn regulates (e.g., decreases) the level of sympathetic nervous system and neurohormonal activation, and increases parasypathetic nervous system activation, thus reducing blood pressure and having a beneficial effect on the cardiovascular system and other body systems. In an embodiment, the present invention provides for assessing the response and the degree to which the baroreflex system of the patient has been responsive to such activation.

The methods, devices, and systems according to the present invention may be used to activate baroreceptors, mechanoreceptors, pressoreceptors, or any other venous heart, or cardiopulmonary receptors which affect the blood pressure, nervous system activity, and neurohormonal activity in a manner analogous to baroreceptors in the arterial vasculation. For convenience, all such venous receptors (and/or nerves carrying signals from such receptors) will be referred to collectively herein as “baroreceptors.”

In an embodiment, the present invention provides methods, devices, and systems for externally applying a baroreflex stimulus to temporarily control/modify a patient's baroreflex behavior. Additionally or alternatively the methods, devices, and systems, also test, evaluate, measure, or confirm a baroreflex response and its extent in a patient in response to the stimulus. Such external stimulation allows a physician to decide how effective an implantable baroreflex activation device would be in a given patient and/or in what location (or locations) to implant such a device. Additionally, such methods, devices, and systems enable baroreflex therapy only for a needed duration of time as for example may be needed in clinical situations such as pregnancy/preeclampsia, acute aortic dissection, and acute hypertensive crisis; as well as shock and acute heart failure.

The methods, devices, and systems of the present invention may be used in a number of manners such as transcutaneously, percutaneously, or surgically. When used in a minimally invasive manner, the methods, devices, and systems of the present invention help physicians and patients avoid unnecessary surgical implantation of baroreflex activation devices.

In some embodiments, the present invention also provides for a number of devices, systems and methods by which the blood pressure, nervous system activity, and neurohormonal activity may be selectively and controllably regulated by activating the baroreflex system. These devices, systems and methods may be implemented, for example, after a physician determines, via the methods and systems just described for external baroreflex activation, that baroreflex activation will provide a desired response in a given patient. By selectively and controllably activating a baroreflex, the present invention reduces excessive blood pressure, sympathetic nervous system activation and neurohormonal activation, thereby minimizing their deleterious effects on the heart, vasculature and other organs and tissues.

In an embodiment of a method embodying features of the present invention for performing a procedure for temporarily modifying baroreflex behavior of a patient includes applying at least a first baroreflex activation stimulus to the patient from a stimulator external to the patient; directing the stimulus through at least one lead configured for temporary placement relative to the patient's body and which is electrically connectable to the external stimulator, and stimulating an area approximating a baroreceptor system of the patient.

In an embodiment, the at least one electrode is disposable within the patient's body. In an embodiment, the lead is configured for transcutaneous placement relative to the patient's body. The lead may be configured for temporary placement within the patient's body. In an embodiment, the lead is detachably connectable to a junction locatable external to the patient's body which is configured for providing electrical communication between the lead and the external pulse generator. The lead may be configured for removal from the patient upon completion of the procedure. In an embodiment, the at least one electrode is surgically disposed within the patient's body and may be configured for removal from the patient upon completion of the procedure. In an embodiment, the at least one electrode is adapted to be removably disposed around a target site at the baroreceptor system of the patient through a primary incision, and is adapted for removal through the primary incision upon completion of the procedure.

In an embodiment, the at least one electrode is percutaneously delivered from a vascular access point to an endovascular target site within the baroreceptor system of the patient. The lead may be adapted for placement exteriorly of the vascular access point.

In many embodiments, the externally applied stimulus comprises some type of transmitted energy. Examples of such transmitted energy include but are not limited to ultrasonic, electromagnetic, radiofrequency and microwave energy. In one embodiment, for example, electromagnetic energy may be transmitted to the patient using at least one electrode external to the patient. In another embodiment, transmitted energy comprises transcutaneous electrical nerve stimulation (TENS). Again, any energy type, form, amount, pattern or the like may be used.

In general, the one or more externally applied baroreflex activation stimuli may be directed toward stimulating a baroreflex via any suitable anatomical structure or structures. In other words, a stimulus may directed at any of a number of various structures to cause baroreflex activation. For example, stimulus may be directed toward one or more carotid sinus nerves, toward one or more carotid baroreceptors, toward other baroreceptors located elsewhere in the body, toward baroreceptor or afferent nerve fibers located in one or more blood vessel walls, toward carotid sinus nerve fibers and/or the like. Thus, the present invention encompasses the application of any external stimulus to activate a baroreflex and is not limited to stimulus of any specific anatomical structure or location. This activation is typically described as “baroreflex activation.” Activation, according to the present invention, may occur directly at, near or in the vicinity of one or more baroreceptors, but is not limited to direct baroreceptor activation. For example, as just mentioned, various nerve fibers may be activated instead of or in addition to baroreceptors.

In an embodiment, to evaluate the response of the patient to the stimulus, the method further includes measuring at least one physiological parameter of the patient, and determining, from the physiological parameter measurement, to what extent the baroreflex activation stimulus caused a baroreflex response in the patient. Generally, the externally applied baroreflex activation stimulus may be any type, form or amount of stimulus. In some embodiments, for example, applying the baroreflex activation stimulus comprises transmitting energy from at least one energy transmitting device, mechanically stimulating an area approximating one or more carotid arteries, and/or introducing one or more drugs into the patient. In an embodiment, the external stimulator is a pulse generator device.

Similarly, any suitable physiological parameter (or multiple parameters) may be measured according to various embodiments of the present invention, for determining whether the applied stimulus has caused baroreflex activation. In various embodiments, for example, parameters which may be measured include but are not limited to blood pressure, change in blood pressure, heart rate, cardiac output, vascular resistance, seizure activity, neurological activity and/or pain sensation. In some embodiments, determining whether baroreflex activation has occurred involves comparing the one or more physiological parameter measurements to one or more baseline measurements. Such a method may optionally involve taking the baseline measurement of the physiological parameter of the patient before externally applying the baroreflex stimulus. Alternatively, one or more threshold measurement levels may be set, and a comparison of the physiological parameter measurements to the threshold(s) may be used to determine whether a baroreflex occurred.

In some embodiments, the at least one physiological parameter measuring device comprises at least one surface electrode for contacting with the patient's skin to measure the physiological parameter. Alternatively, the physiological parameter measuring device may comprise at least one piezoelectric sensor for contacting with the patient's skin to measure the physiological parameter. In other embodiments, the measuring device may comprise a blood pressure cuff, a pulse oximetry device, a Swan-Ganz catheter a device for measuring cardiac output, a device for measuring vascular resistance, electroencephalogram device and/or the like. Any suitable measuring device or combination of devices, either now known or hereafter discovered, may be used without departing from the scope of the present invention. Such devices may be used to measure any suitable physiological parameter or parameters, such as but not limited to blood pressure, change in blood pressure, heart rate, cardiac output, vascular resistance, seizure activity, neurological activity and/or pain sensation.

In some embodiments, the system may also include a processor for receiving physiological parameter measurements from the measuring device and processing the measurements into data in a usable form. For example, such a processor may compare measured physiological parameter data to one or more baseline measurement values to determine whether the applied stimulus has caused baroreflex activation in the patient. In some embodiments, the system may further include a display monitor coupled with the processor for displaying measured physiological parameter data to a user.

It should be appreciated that methods, devices, and systems according to the present invention may be used alone or in combination with other therapy methods and devices to achieve separate, complementary, or synergistic effects. Examples of such other methods and devices include Cardiac resynchronization therapy (CRT), Cardiac Rhythm Management (CRM), anti-arrhythmia treatment as for example applied to the heart via a cardioverter/defibrillator; drug delivery devices (e.g., drug pump) and systems; neurostimulators, as well as diagnostic and/or monitoring modalities. The above devices and/or systems, may be separate or integrated into a combination device in which the component therapies perform independently or in concert.

These and other aspects and embodiments of the present invention are described in further detail below.

### BRIEF DESCRIPTION OF THE DRAWINGS

FIG. 1 is a schematic illustration of the upper torso of a human body, showing the major arteries and veins and associated anatomy.

FIG. 2A is a cross-sectional schematic illustration of the carotid sinus and baroreceptors within the vascular wall.

FIG. 2B is a schematic illustration of baroreceptors within the vascular wall and the baroreflex system.

FIG. 3 is a schematic illustration of the upper torso of a human body, demonstrating a system for externally applying a baroreflex activation stimulus to the body and measuring a physiological parameter.

FIG. 4 is a schematic illustration of a baroreflex activation system in accordance with the present invention.

FIGS. 5A and 5B are schematic illustrations of a baroreflex activation device in the form of an implantable extraluminal conductive structure which electrically induces a baroreceptor signal in accordance with an embodiment of the present invention.

FIGS. 6A-6F are schematic illustrations of various possible arrangements of electrodes around the carotid sinus for extravascular electrical activation embodiments.

FIG. 7 is a schematic illustration of a system including an external controller connected to an implanted baroreflex activation device by way of a transcutaneous lead.

### DETAILED DESCRIPTION OF THE INVENTION

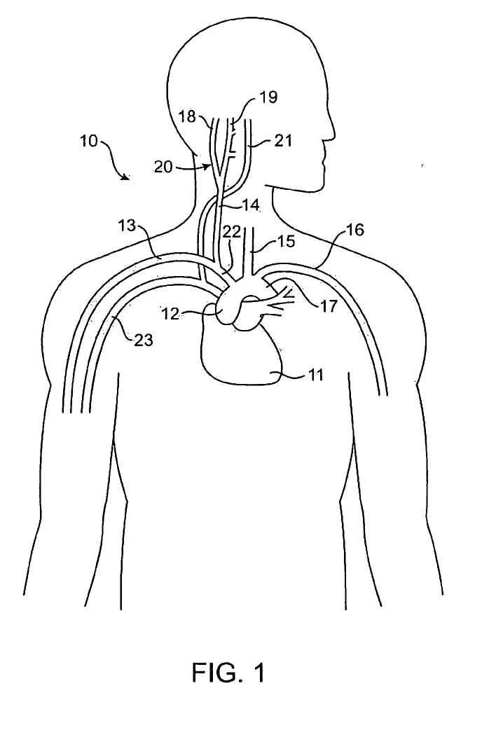

To better understand the present invention, it may be useful to explain some of the basic vascular anatomy associated with the cardiovascular system. Referring to FIG. 1, a schematic illustration of the upper torso of a human body 10 shows some of the major arteries and veins of the cardiovascular system. The left ventricle of the heart 11 pumps oxygenated blood up into the aortic arch 12. The right subclavian artery 13, the right common carotid artery 14, the left common carotid artery 15 and the left subclavian artery 16 branch off the aortic arch 12 proximal of the descending thoracic aorta 17. Although relatively short, a distinct vascular segment referred to as the brachiocephalic artery 22 connects the right subclavian artery 13 and the right common carotid artery 14 to the aortic arch 12. The right carotid artery 14 bifurcates into the right external carotid artery 18 and the right internal carotid artery 19 at the right carotid sinus 20. Although not shown for purposes of clarity only, the left carotid artery 15 similarly bifurcates into the left external carotid artery and the left internal carotid artery at the left carotid sinus.

From the aortic arch 12, oxygenated blood flows into the carotid arteries 18/19 and the subclavian arteries 13/16. From the carotid arteries 18/19, oxygenated blood circulates through the head and cerebral vasculature and oxygen depleted blood returns to the heart 11 by way of the jugular veins, of which only the right internal jugular vein 21 is shown for sake of clarity. From the subclavian arteries 13/16, oxygenated blood circulates through the upper peripheral vasculature and oxygen depleted blood returns to the heart by way of the subclavian veins, of which only the right subclavian vein 23 is shown, also for sake of clarity. The heart 11 pumps the oxygen depleted blood through the pulmonary system where it is re-oxygenated. The re-oxygenated blood returns to the heart 11 which pumps the re-oxygenated blood into the aortic arch as described above, and the cycle repeats.

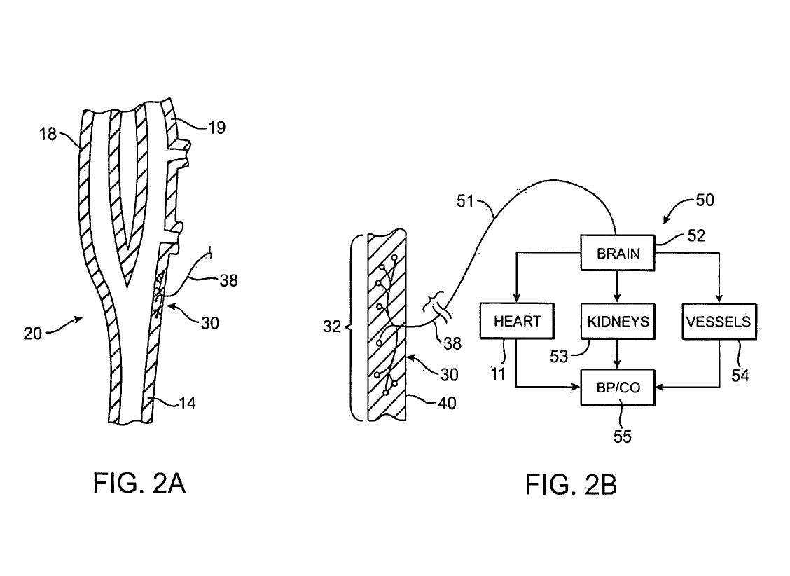

Within the arterial walls of the aortic arch 12, common carotid arteries 14/15 (near the right carotid sinus 20 and left carotid sinus), subclavian arteries 13/16 and brachiocephalic artery 22 there are baroreceptors 30. For example, as best seen in FIG. 2A, baroreceptors 30 reside within the vascular walls of the carotid sinus 20. Baroreceptors 30 are a type of stretch receptor used by the body to sense blood pressure. In general, the term “baroreceptors” may refer to baroreceptors themselves as well as other receptors that act like baroreceptors. An increase in blood pressure causes the arterial wall to stretch, and a decrease in blood pressure causes the arterial wall to return to its original size. Such a cycle is repeated with each beat of the heart. Because baroreceptors 30 are located within the arterial wall, they are able to sense deformation of the adjacent tissue, which is indicative of a change in blood pressure. The baroreceptors 30 located in the right carotid sinus 20, the left carotid sinus and the aortic arch 12 may play the most significant role in sensing blood pressure that affects the baroreflex system 50, which is described in more detail with reference to FIG. 2B.

Referring now to FIG. 2B, which shows a schematic illustration of baroreceptors 30 disposed in a generic vascular wall 40 and a schematic flow chart of the baroreflex system 50. Baroreceptors 30 are profusely distributed within the arterial walls 40 of the major arteries discussed previously, and generally form an arbor 32. The baroreceptor arbor 32 comprises a plurality of baroreceptors 30, each of which transmits baroreceptor signals to the brain 52 via nerve 38. The baroreceptors 30 are so profusely distributed and arborized within the vascular wall 40 that discrete baroreceptor arbors 32 are not readily discernable. To this end, the baroreceptors 30 shown in FIG. 2B are primarily schematic for purposes of illustration.

Baroreflex signals are used to activate a number of body systems which collectively may be referred to as the baroreflex system 50. Baroreceptors 30 (and other baroreceptor-like receptors) are connected to the brain 52 via the nervous system 51. Thus, the brain 52 is able to detect changes in blood pressure, which is indicative of cardiac output. If cardiac output is insufficient to meet demand (i.e., the heart 11 is unable to pump sufficient blood), the baroreflex system 50 activates a number of body systems, including the heart 11, kidneys 53, vessels 54, and other organs/tissues. Such activation of the baroreflex system 50 generally corresponds to an increase in neurohormonal activity. Specifically, the baroreflex system 50 initiates a neurohormonal sequence that signals the heart 11 to increase heart rate and increase contraction force in order to increase cardiac output, signals the kidneys 53 to increase blood volume by retaining sodium and water, and signals the vessels 54 to constrict to elevate blood pressure. The cardiac, renal and vascular responses increase blood pressure and cardiac output 55, and thus increase the workload of the heart 11. Conversely, if a patient's blood pressure is elevated, the opposite baroreflex response typically occurs.

To address the problems of hypertension, heart failure, other cardiovascular disorders and renal disorders, the present invention provides a number of devices, systems and methods by which the baroreflex system 50 is activated to reduce excessive blood pressure, autonomic nervous system activity and neurohormonal activation. Although much of the following description focuses on use of baroreflex activation to treat cardiovascular conditions, however, the invention is in no way limited to such applications. In fact, according to various embodiments, baroreceptor activation may be used for any other suitable purpose, such as for controlling seizure activity to treat epilepsy (described fully in U.S. Patent Application Ser. No. 60/505,121 (Attorney Docket No. 021433-000900US), filed Sep. 22, 2003) or for pain control and/or sedation (described fully in U.S. Patent Application Ser. No. 60/513,642 (Attorney Docket No. 021433-001000US), filed Oct. 22, 2003). Other embodiments may involve baroreflex activation for any other suitable purpose.

In particular, the present invention provides a number of devices, systems and methods by which baroreceptors 30 and other baroreflex structures may be activated, thereby indicating an increase in blood pressure and signaling the brain 52 to reduce the body's blood pressure and level of sympathetic nervous system and neurohormonal activation, and increase parasypathetic nervous system activation, thus having a beneficial effect on the cardiovascular system and other body systems. In an embodiment, according to the present invention the baroreceptors are activated by way of an external stimulus generator. In an embodiment, the stimulation is for a temporary period of time. As was previously discussed, various embodiments of the present invention may operate by activating baroreceptors 30, other receptors, nerve fibers connected to one or more baroreceptors, such as carotid sinus nerve fibers, or any other suitable structure for causing a baroreflex, and activation may be provided directly at a structure or in the vicinity of a structure. This type of activation is generally referred to herein as “baroreflex activation.” For convenience, the phrase “activating baroreceptors” may often be used to generally refer to activating any of the structures just mentioned for causing baroreflex activation.

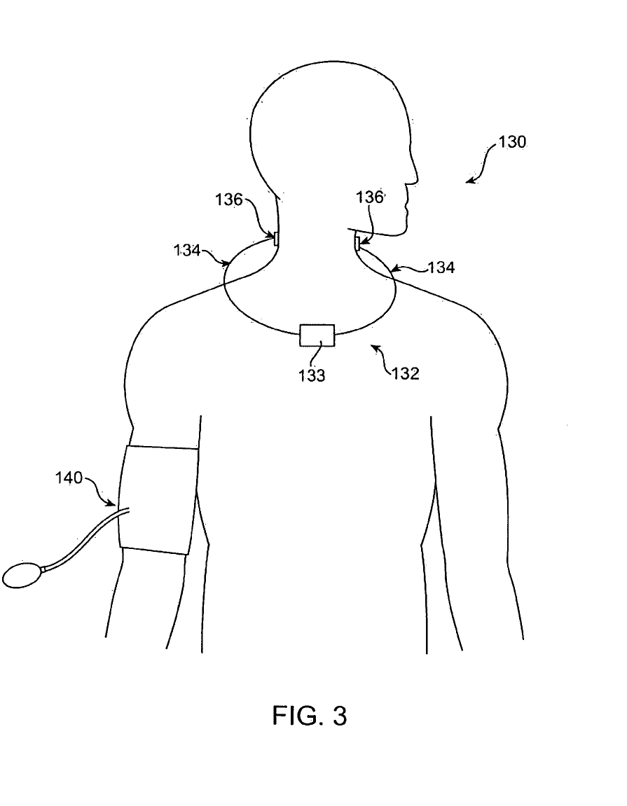

With reference now to FIG. 3, the present invention generally provides a device 129 for externally applying a stimulus to a patient 130 to invoke a baroreflex response, including at least one external baroreflex activation device 132 externally located to the patient 30. In the embodiment, as shown, the external stimulus generating device includes a controller 133 having a pulse generator electronically connected to two leads 134 extending from either side of the controller 133. The leads are located transcutaneously relative to the patient in the embodiment shown. Stimulus is carried from the controller 133 through the leads 134 to electrodes 136. In an embodiment, there is at least one interface for electrically connecting the stimulus generator to the leads 134, as for example, 137. In an embodiment as shown the interface 137 is configured for permanent or removable attachment from either or both the stimulator 133 and the lead 134 which is connected to the interface 137.

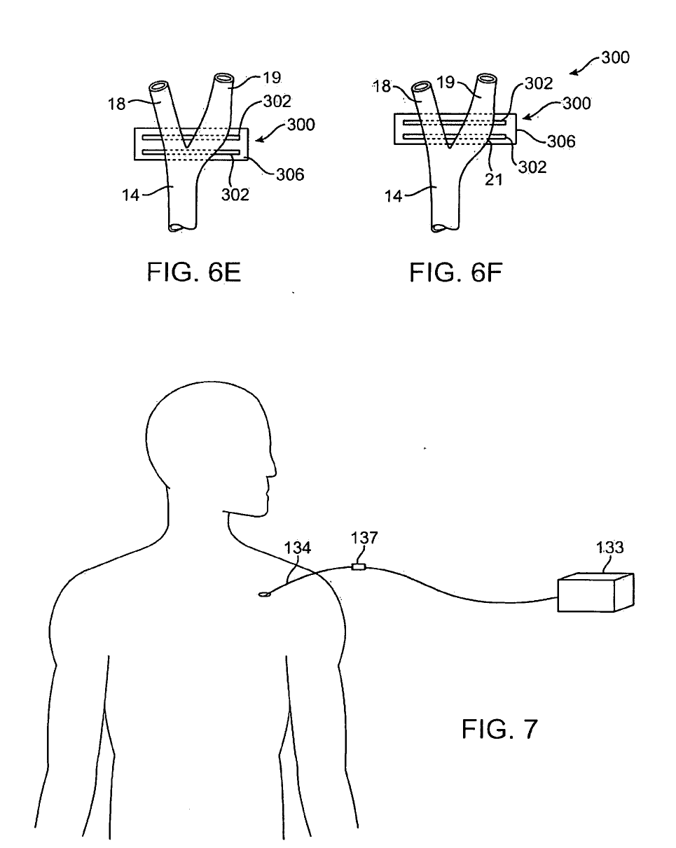

In an embodiment, at least one temporary, percutaneous lead delivers stimulus from the external stimulator 133 to the patient through the electrodes 136. The electrode 136 may be located within the patient's body. The placement of the electrode 136 may be achieved in a number of ways, and it may be placed temporarily or permanently. In one embodiment depicted in FIG. 7, the external controller 133 is coupled to lead 134 via interface 137. Lead 134 passes transcutaneously, through the skin of the patient, to a baroreflex activation device implanted within the patient.

In an embodiment, the electrode may be surgically disposed within the patient's body at a suitable target site, as discussed below. Such electrode, may be disposed within the patient's body through a primary incision point and held in place without the use of any sutures such that upon the completion of the desired period of time (e.g., temporary duration or upon measuring and/or assessing the effect of such baroreflex activation) it may be removed from the same primary incision point. In an embodiment, the electrode may be an endovascular electrode which is delivered to the target site percutaneously through an access point (e.g., femoral artery). In such an embodiment, the endovascular electrode, may similarly be removable from the patient's body upon the completion of the desired period of time. Alternatively, the same or a different electrode as part of a permanently disposable baroreflex activation device may be disposed within the patient's body.

In an embodiment, the present invention may be used to invoke a baroreflex and measuring one or more physiological parameters. The measured parameter(s) may then be used to determine to what extent the applied stimulus caused a baroreflex, thus providing a physician with information as to the efficacy an implantable baroreflex activation device will have in a given patient. Generally, a baroreflex activation/measuring system includes at least one baroreflex activation device 132 and at least one physiological parameter measuring device 140. In various embodiments, activation device 132 may comprise, for example, an energy transmission device, a mechanical force application device for applying massage to a carotid artery, a drug delivery device for delivering one or more drugs to patient 130 to elicit a baroreflex and/or the like. Any suitable device or combination of devices may be used. In FIG. 3, activation device 132 comprises an energy electromagnetic energy source 133 coupled with two electrodes 136 via two leads 134. Alternatively, energy source 133 may comprise an ultrasound energy source, microwave energy source, TENS unit, RF energy source or a source of any other suitable energy. Electrodes 136 may alternatively comprise any other energy transmission members, such as ultrasound transmission members or the like.

Although electrodes 136 are shown coupled with the patient's 130 neck, they could alternatively be placed at any other suitable location for activating a baroreflex. For example, they could be coupled with the patient near another location where baroreceptors or baroreceptor nerves are present. Alternatively, one or more energy transmission members may be positioned so as to not contact the patient. Any number of energy transmission members may be used, with some embodiments including only one and other including multiple energy transmission units. And as mentioned, other modalities may be used for activating a baroreflex, such as mechanical stimulation, drug activation and/or the like.

Measuring device 140 may similarly include any suitable device or combination of devices. In the embodiment shown, measuring device 140 is a sphygmomanometer, but any other suitable device may be used, such as a pulse oximeter, a Swan-Ganz catheter, an ECG or EEG device, or the like. Any parameter indicative of a baroreflex may be measured, such as blood pressure, change in blood pressure, heart rate, cardiac output, vascular resistance, seizure activity, neurological activity, pain sensation, patient sedation and/or the like. Using measuring device 140, a physician may determine the extent to which a baroreflex has been caused by application of a stimulus by activation device 132, and thus may determine whether an implantable activation device will achieve a desired result. In some instances, a physician may decide that baroreflex activation is not desirable in a given patient and will thus decide not to implant an activation device.

In some embodiments, multiple baroreflex stimuli may be applied to a patient and the resulting baroreflex activations after application of the stimuli can be compared. For example, stimuli of different intensities and/or applied from different locations may be compared and data describing the results of those stimuli may be provided to a physician. The physician may then use the data to choose an optimal or desirable location(s) for placing one or more implantable activators and/or to choose an intensity at which to set the activator(s). To facilitate such a process, in some embodiments a system may further include a processor for processing measurements taken by measuring device 140 and/or a monitor or other read-out mechanism for providing useful data to a physician user.

Once a physician determines that a given patient will respond favorably to an implanted baroreflex stimulation device, the next step may be to actually implant such a device. The following description focuses on a number of implantable devices for baroreflex activation. However, the invention is in no way limited to use of the implantable devices described below. In fact, any suitable implantable device may optionally be used as part of a method or system of the present invention. In some embodiments, for example, an implantable device or system may also include one or more external components or parts, which are disposed outside the patient's body during treatment.

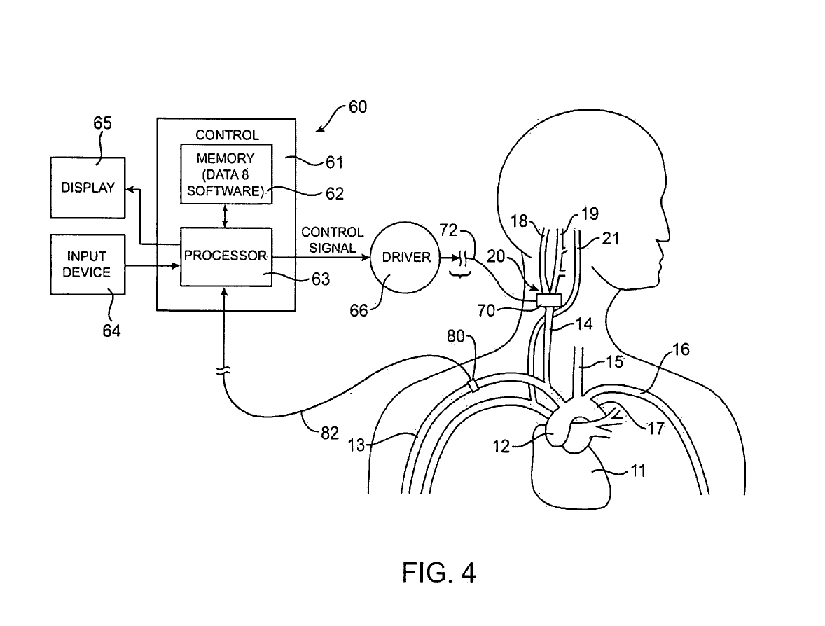

That being said, and with reference now to FIG. 4, the present invention generally provides a system including a control system 60, a baroreflex activation device 70, and a sensor 80 (optional), which generally operate in the following manner. The sensor(s) 80 optionally senses and/or monitors a parameter (e.g., cardiovascular function) indicative of the need to modify the baroreflex system and generates a signal indicative of the parameter. The control system 60 generates a control signal as a function of the received sensor signal. The control signal activates, deactivates or otherwise modulates the baroreflex activation device 70. Typically, activation of the device 70 results in activation of the baroreceptors 30 (or other baroreflex structures). Alternatively, deactivation or modulation of the baroreflex activation device 70 may cause or modify activation of the baroreceptors 30. The baroreflex activation device 70 may comprise a wide variety of devices which utilize electrical means to activate baroreceptors 30. Thus, when the sensor 80 detects a parameter indicative of the need to modify the baroreflex system activity (e.g., excessive blood pressure), the control system 60 generates a control signal to modulate (e.g. activate) the baroreflex activation device 70 thereby inducing a baroreflex signal that is perceived by the brain 52 to be apparent excessive blood pressure. When the sensor 80 detects a parameter indicative of normal body function (e.g., normal blood pressure), the control system 60 generates a control signal to modulate (e.g., deactivate) the baroreflex activation device 70.

As mentioned previously, the baroreflex activation device 70 may comprise a wide variety of devices which utilize electrical means to activate the baroreceptors 30. The baroreflex activation device 70 of the present invention comprises an electrode structure which directly activates one or more baroreceptors 30 by changing the electrical potential across the baroreceptors 30. It is possible that changing the electrical potential across the tissue surrounding the baroreceptors 30 may cause the surrounding tissue to stretch or otherwise deform, thus mechanically activating the baroreceptors 30, in which case the stretchable and elastic electrode structures of the present invention may provide significant advantages.

All of the specific embodiments of the electrode structures of the present invention are suitable for implantation, and are preferably implanted using a minimally invasive surgical approach. The baroreflex activation device 70 may be positioned anywhere baroreceptors 30 are present. Such potential implantation sites are numerous, such as the aortic arch 12, in the common carotid arteries 18/19 near the carotid sinus 20, in the subclavian arteries 13/16, in the brachiocephalic artery 22, or in other arterial or venous locations. The electrode structures of the present invention will be implanted such that they are positioned on or over a vascular structure at or near the baroreceptors 30. Preferably, the electrode structure of the baroreflex activation device 70 is implanted near the right carotid sinus 20 and/or the left carotid sinus (near the bifurcation of the common carotid artery) and/or the aortic arch 12, where baroreceptors 30 have a significant impact on the baroreflex system 50. For purposes of illustration only, the present invention is described with reference to baroreflex activation device 70 positioned near the carotid sinus 20.

The optional sensor 80 is operably coupled to the control system 60 by electric sensor cable or lead 82. The sensor 80 may comprise any suitable device that measures or monitors a parameter indicative of the need to modify the activity of the baroreflex system. For example, the sensor 80 may comprise a physiologic transducer or gauge that measures ECG, blood pressure (systolic, diastolic, average or pulse pressure), blood volumetric flow rate, blood flow velocity, blood pH, O2 or CO2 content, mixed venous oxygen saturation (SVO2), vasoactivity, nerve activity, tissue activity, body movement, activity levels, respiration, or composition. Examples of suitable transducers or gauges for the sensor 80 include ECG electrodes, a piezoelectric pressure transducer, an ultrasonic flow velocity transducer, an ultrasonic volumetric flow rate transducer, a thermodilution flow velocity transducer, a capacitive pressure transducer, a membrane pH electrode, an optical detector (SVO2), tissue impedance (electrical), or a strain gauge. Although only one sensor 80 is shown, multiple sensors 80 of the same or different type at the same or different locations may be utilized.

An example of an implantable blood pressure measurement device that may be disposed about a blood vessel is disclosed in U.S. Pat. No. 6,106,477 to Miesel et al., the entire disclosure of which is incorporated herein by reference. An example of a subcutaneous ECG monitor is available from Medtronic under the trade name REVEAL ILR and is disclosed in PCT Publication No. WO 98/02209, the entire disclosure of which is incorporated herein by reference. Other examples are disclosed in U.S. Pat. Nos. 5,987,352 and 5,331,966, the entire disclosures of which are incorporated herein by reference. Examples of devices and methods for measuring absolute blood pressure utilizing an ambient pressure reference are disclosed in U.S. Pat. No. 5,810,735 to Halperin et al., U.S. Pat. No. 5,904,708 to Goedeke, and PCT Publication No. WO 00/16686 to Brockway et al., the entire disclosures of which are incorporated herein by reference. The sensor 80 described herein may take the form of any of these devices or other devices that generally serve the same purpose.

The sensor 80 may be positioned in/on a major artery such as the aortic arch 12, a common carotid artery 14/15, a subclavian artery 13/16 or the brachiocephalic artery 22, or in a chamber of the heart 11, such that the parameter of interest may be readily ascertained. The sensor 80 may be disposed inside the body such as in or on an artery, a vein or a nerve (e.g. vagus nerve), or disposed outside the body, depending on the type of transducer or gauge utilized. The sensor 80 may be separate from the baroreflex activation device 70 or combined therewith. For purposes of illustration only, the sensor 80 is shown positioned on the right subclavian artery 13.

By way of example, the control system 60 includes a control block 61 comprising a processor 63 and a memory 62. Control system 60 is connected to the sensor 80 by way of sensor cable 82. Control system 60 is also connected to the baroreflex activation device 70 by way of electric control cable 72. Thus, the control system 60 receives a sensor signal from the sensor 80 by way of sensor cable 82, and transmits a control signal to the baroreflex activation device 70 by way of control cable 72.

The system components 60/70/80 may be directly linked via cables 72/82 or by indirect means such as RF signal transceivers, ultrasonic transceivers or galvanic couplings. Examples of such indirect interconnection devices are disclosed in U.S. Pat. No. 4,987,897 to Funke and U.S. Pat. No. 5,113,859 to Funke, the entire disclosures of which are incorporated herein by reference.

The memory 62 may contain data related to the sensor signal, the control signal, and/or values and commands provided by the input device 64. The memory 62 may also include software containing one or more algorithms defining one or more functions or relationships between the control signal and the sensor signal. The algorithm may dictate activation or deactivation control signals depending on the sensor signal or a mathematical derivative thereof. The algorithm may dictate an activation or deactivation control signal when the sensor signal falls below a lower predetermined threshold value, rises above an upper predetermined threshold value or when the sensor signal indicates a specific physiologic event. The algorithm may dynamically alter the threshold value as determined by the sensor input values.

As mentioned previously, the baroreflex activation device 70 activates baroreceptors 30 and/or other baroreflex structures electrically, optionally in combination with mechanical, thermal, chemical, biological or other co-activation. In some instances, the control system 60 includes a driver 66 to provide the desired power mode for the baroreflex activation device 70. For example, the driver 66 may comprise a power amplifier or the like and the cable 72 may comprise electrical lead(s). In other instances, the driver 66 may not be necessary, particularly if the processor 63 generates a sufficiently strong electrical signal for low level electrical actuation of the baroreflex activation device 70.

The control system 60 may operate as a closed loop utilizing feedback from the sensor 80, or other sensors, such as heart rate sensors which may be incorporated or the electrode assembly, or as an open loop utilizing reprogramming commands received by input device 64. The closed loop operation of the control system 60 preferably utilizes some feedback from the transducer 80, but may also operate in an open loop mode without feedback. Programming commands received by the input device 64 may directly influence the control signal, the output activation parameters, or may alter the software and related algorithms contained in memory 62. The treating physician and/or patient may provide commands to input device 64. Display 65 may be used to view the sensor signal, control signal and/or the software/data contained in memory 62.

The control signal generated by the control system 60 may be continuous, periodic, alternating, episodic or a combination thereof, as dictated by an algorithm contained in memory 62. Continuous control signals include a constant pulse, a constant train of pulses, a triggered pulse and a triggered train of pulses. Examples of periodic control signals include each of the continuous control signals described above which have a designated start time (e.g., beginning of each period as designated by minutes, hours, or days in combinations of) and a designated duration (e.g., seconds, minutes, hours, or days in combinations of). Examples of alternating control signals include each of the continuous control signals as described above which alternate between the right and left output channels. Examples of episodic control signals include each of the continuous control signals described above which are triggered by an episode (e.g., activation by the physician/patient, an increase/decrease in blood pressure above a certain threshold, heart rate above/below certain levels, etc.).

The stimulus regimen governed by the control system 60 may be selected to promote long term efficacy. It is theorized that uninterrupted or otherwise unchanging activation of the baroreceptors 30 may result in the baroreceptors and/or the baroreflex system becoming less responsive over time, thereby diminishing the long term effectiveness of the therapy. Therefore, the stimulus regimen may be selected to activate, deactivate or otherwise modulate the baroreflex activation device 70 in such a way that therapeutic efficacy is maintained preferably for years.

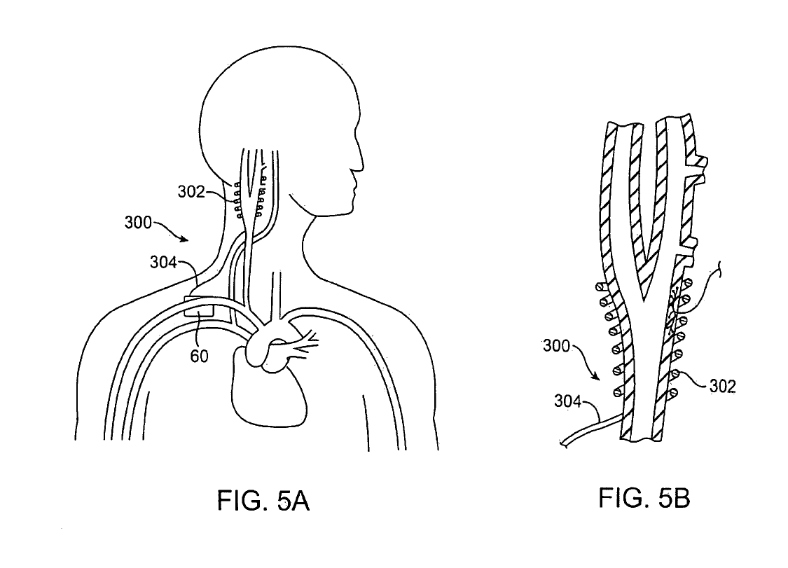

FIGS. 5A and 5B show schematic illustrations of a baroreflex activation device 300 in the form of an extravascular electrically conductive structure or electrode 302. The electrode structure 302 may comprise a coil, braid or other structure capable of surrounding the vascular wall. Alternatively, the electrode structure 302 may comprise one or more electrode patches distributed around the outside surface of the vascular wall. Because the electrode structure 302 is disposed on the outside surface of the vascular wall, intravascular delivery techniques may not be practical, but minimally invasive surgical techniques will suffice. The extravascular electrode structure 302 may receive electrical signals directly from the driver 66 of the control system 60 by way of electrical lead 304, or indirectly by utilizing an inductor (not shown) as described in commonly assigned application Ser. No. 10/402,393, previously incorporated by reference.

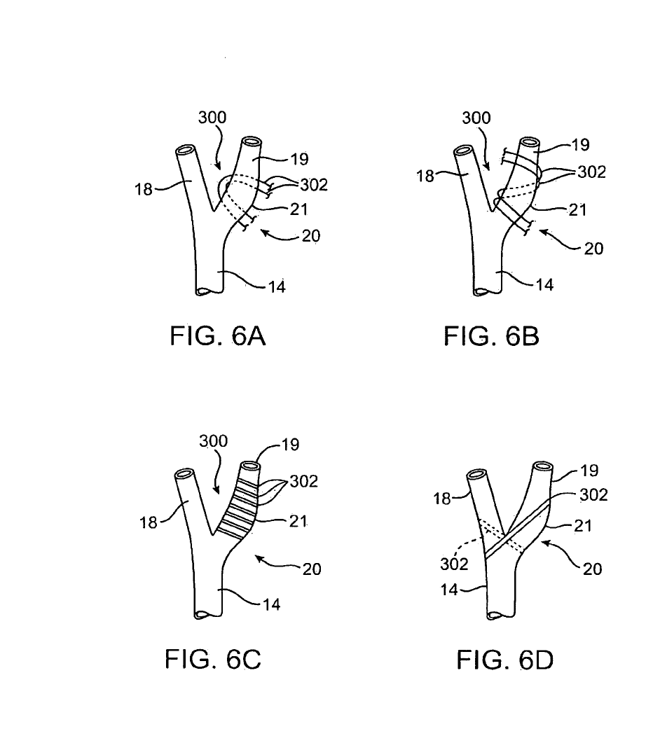

Refer now to FIGS. 6A-6F which show schematic illustrations of various possible arrangements of electrodes around the carotid sinus 20 for extravascular electrical activation embodiments, such as baroreflex activation device 300 described with reference to FIGS. 4A and 4B. The electrode designs illustrated and described hereinafter may be particularly suitable for connection to the carotid arteries at or near the carotid sinus, and may be designed to minimize extraneous tissue stimulation.

In FIGS. 6A-6F, the carotid arteries are shown, including the common 14, the external 18 and the internal 19 carotid arteries. The location of the carotid sinus 20 may be identified by a landmark bulge 21, which is typically located on the internal carotid artery 19 just distal of the bifurcation, or extends across the bifurcation from the common carotid artery 14 to the internal carotid artery 19.

The carotid sinus 20, and in particular the bulge 21 of the carotid sinus, may contain a relatively high density of baroreceptors 30 (not shown) in the vascular wall. For this reason, it may be desirable to position the electrodes 302 of the activation device 300 on and/or around the sinus bulge 21 to maximize baroreceptor responsiveness and to minimize extraneous tissue stimulation.

It should be understood that the device 300 and electrodes 302 are merely schematic, and only a portion of which may be shown, for purposes of illustrating various positions of the electrodes 302 on and/or around the carotid sinus 20 and the sinus bulge 21. In each of the embodiments described herein, the electrodes 302 may be monopolar, bipolar, or tripolar (anode-cathode-anode or cathode-anode-cathode sets). Specific extravascular electrode designs are described in more detail hereinafter.

In FIG. 6A, the electrodes 302 of the extravascular electrical activation device 300 extend around a portion or the entire circumference of the sinus 20 in a circular fashion. Often, it would be desirable to reverse the illustrated electrode configuration in actual use. In FIG. 6B, the electrodes 302 of the extravascular electrical activation device 300 extend around a portion or the entire circumference of the sinus 20 in a helical fashion. In the helical arrangement shown in FIG. 6B, the electrodes 302 may wrap around the sinus 20 any number of times to establish the desired electrode 302 contact and coverage. In the circular arrangement shown in FIG. 6A, a single pair of electrodes 302 may wrap around the sinus 20, or a plurality of electrode pairs 302 may be wrapped around the sinus 20 as shown in FIG. 6C to establish more electrode 302 contact and coverage.

The plurality of electrode pairs 302 may extend from a point proximal of the sinus 20 or bulge 21, to a point distal of the sinus 20 or bulge 21 to ensure activation of baroreceptors 30 throughout the sinus 20 region. The electrodes 302 may be connected to a single channel or multiple channels as discussed in more detail hereinafter. The plurality of electrode pairs 302 may be selectively activated for purposes of targeting a specific area of the sinus 20 to increase baroreceptor responsiveness, or for purposes of reducing the exposure of tissue areas to activation to maintain baroreceptor responsiveness long term.

In FIG. 6D, the electrodes 302 extend around the entire circumference of the sinus 20 in a criss-cross fashion. The criss-cross arrangement of the electrodes 302 establishes contact with both the internal 19 and external 18 carotid arteries around the carotid sinus 20. Similarly, in FIG. 6E, the electrodes 302 extend around all or a portion of the circumference of the sinus 20, including the internal 19 and external 18 carotid arteries at the bifurcation, and in some instances the common carotid artery 14. In FIG. 6F, the electrodes 302 extend around all or a portion of the circumference of the sinus 20, including the internal 19 and external 18 carotid arteries distal of the bifurcation. In FIGS. 6E and 6F, the extravascular electrical activation devices 300 are shown to include a substrate or base structure 306 which may encapsulate and insulate the electrodes 302 and may provide a means for attachment to the sinus 20 as described in more detail hereinafter.

From the foregoing discussion with reference to FIGS. 6A-6F, it should be apparent that there are a number of suitable arrangements for the electrodes 302 of the activation device 300, relative to the carotid sinus 20 and associated anatomy. In each of the examples given above, the electrodes 302 are wrapped around a portion of the carotid structure, which may require deformation of the electrodes 302 from their relaxed geometry (e.g., straight). To reduce or eliminate such deformation, the electrodes 302 and/or the base structure 306 may have a relaxed geometry that substantially conforms to the shape of the carotid anatomy at the point of attachment. In other words, the electrodes 302 and the base structure or backing 306 may be pre shaped to conform to the carotid anatomy in a substantially relaxed state. Alternatively, the electrodes 302 may have a geometry and/or orientation that reduces the amount of electrode 302 strain. Optionally, as described in more detail below, the backing or base structure 306 may be elastic or stretchable to facilitate wrapping of and conforming to the carotid sinus or other vascular structure.

Refer now to FIG. 13 which schematically illustrates an extravascular electrical activation device 300 including a support collar or anchor 312. In this embodiment, the activation device 300 is wrapped around the internal carotid artery 19 at the carotid sinus 20, and the support collar 312 is wrapped around the common carotid artery 14. The activation device 300 is connected to the support collar 312 by cables 304, which act as a loose tether. With this arrangement, the collar 312 isolates the activation device from movements and forces transmitted by the cables 304 proximal of the support collar, such as may be encountered by movement of the control system 60 and/or driver 66. As an alternative to support collar 312, a strain relief (not shown) may be connected to the base structure 306 of the activation device 300 at the juncture between the cables 304 and the base 306. With either approach, the position of the device 300 relative to the carotid anatomy may be better maintained despite movements of other parts of the system.

In this embodiment, the base structure 306 of the activation device 300 may comprise molded tube, a tubular extrusion, or a sheet of material wrapped into a tube shape utilizing a suture flap 308 with sutures 309 as shown. The base structure 306 may be formed of a flexible and biocompatible material such as silicone, which may be reinforced with a flexible material such as polyester fabric available under the trade name DACRON® to form a composite structure. The inside diameter of the base structure 306 may correspond to the outside diameter of the carotid artery at the location of implantation, for example 6 to 8 mm. The wall thickness of the base structure 306 may be very thin to maintain flexibility and a low profile, for example less than 1 mm. If the device 300 is to be disposed about a sinus bulge 21, a correspondingly shaped bulge may be formed into the base structure for added support and assistance in positioning.

The support collar 312 may be formed similarly to base structure 306. For example, the support collar may comprise molded tube, a tubular extrusion, or a sheet of material wrapped into a tube shape utilizing a suture flap 315 with sutures 313 as shown. The support collar 312 may be formed of a flexible and biocompatible material such as silicone, which may be reinforced to form a composite structure. The cables 304 are secured to the support collar 312, leaving slack in the cables 304 between the support collar 312 and the activation device 300.

In all embodiments described herein, it may be desirable to secure the activation device to the vascular wall using sutures or other fixation means. For example, sutures 311 may be used to maintain the position of the electrical activation device 300 relative to the carotid anatomy (or other vascular site containing baroreceptors, nerve fibers or the like). Such sutures 311 may be connected to base structure 306, and pass through all or a portion of the vascular wall. For example, the sutures 311 may be threaded through the base structure 306, through the adventitia of the vascular wall, and tied. If the base structure 306 comprises a patch or otherwise partially surrounds the carotid anatomy, the corners and/or ends of the base structure may be sutured, with additional sutures evenly distributed therebetween. In order to minimize the propagation of a hole or a tear through the base structure 306, a reinforcement material such as polyester fabric may be embedded in the silicone material. In addition to sutures, other fixation means may be employed such as staples or a biocompatible adhesive, for example.

In most activation device embodiments described herein, it may be desirable to incorporate anti-inflammatory agents (e.g., steroid eluting electrodes) such as described in U.S. Pat. No. 4,711,251 to Stokes, U.S. Pat. No. 5,522,874 to Gates and U.S. Pat. No. 4,972,848 to Di Domenico et al., the entire disclosures of which are incorporated herein by reference. Such agents reduce tissue inflammation at the chronic interface between the device (e.g., electrodes) and the vascular wall tissue, to thereby increase the efficiency of stimulus transfer, reduce power consumption, and maintain activation efficiency, for example.

Any of the devices described above may be used alone or with other compatible devices. In some embodiments, in fact, an implantable device for baroreceptor activation may be incorporated with another implantable device for performing a related or entirely different function. For example, it may be advantageous to incorporate a baroreflex activation device as described above with an implantable cardiac pacemaker such as a bi-ventricular pacing device, defibrillator, cardioverter defibrillator, a drug pump, a neurostimulator and/or the like. Thus, it is contemplated within the scope of the invention that various devices for providing baroreflex activation may be suitable for use with or incorporation into any other suitable implantable device.

Additional disclosure material that exemplifies at least a portion of the other features and functionality of the range of embodiments within the spirit and scope of the present invention can be found in U.S. Pat. No. 6,522,926 to Kieval et al. and U.S. Published Patent Application No. 2006/0293712 to Kieval et al., the disclosures of which are hereby incorporated by reference in their entireties.

The present invention may be manifested in a variety of forms other than the specific embodiments described and contemplated herein. Accordingly, departures in form and detail may be made without departing from the scope and spirit of the present invention as described in the appended claims.

What is claimed is:

1. A baroreflex therapy system for providing temporary and chronic treatments to a patient, comprising:
an implantable baroreflex activation device having at least one electrode and including a lead with a first end coupled to the at least one electrode and a second end with a connector;
an external controller, including a pulse generator adapted to deliver baroreflex therapy pulses to the implantable baroreflex activation device via a cable having one end adapted to releasably couple with the connector of the lead of the implantable baroreflex activation device, the controller adapted to activate, deactivate or otherwise modulate the implantable baroreflex activation device based on a signal received from a sensor; and
an implantable baroreflex activation therapy pulse generator including a header adapted to connect with the connector,
the system being configured to determine based on a first temporary period whether the patient is responding to the baroreflex therapy as generated by the external controller, and in response, provide an indication that the implantable baroreflex activation therapy pulse generator should be implanted and connected to the implantable baroreflex activation device to provide baroreflex for a second chronic period.

2. The system of claim 1, wherein the sensor is configured to sense blood volume.

3. The system of claim 1, wherein the baroreflex activation device is configured to be implanted intravascularly.

4. The system of claim 1, wherein the baroreflex activation device is configured to be implanted extravascularly.

5. A method of treating a patient, comprising:
providing a baroreflex activation device having at least one electrode;
providing an external controller, including a pulse generator and a junction;
providing a lead coupled to the baroreflex activation device, the lead including a distal end configured to be releasably coupled to the junction;
providing instructions to treat the patient, including:
implanting the baroreflex activation device proximate a baroreceptor in a vascular wall;
positioning the distal end of the lead through the skin of the patient such that the distal end of the lead is outside the body of the patient;
releasably coupling the lead to the controller; and
activating, deactivating, or otherwise modulating the at least one electrode with the controller to cause a baroreflex in the patient.

6. The method of claim 5, further comprising:
providing a sensor;
sensing a patient physiological parameter with the sensor;
generating a sensor signal indicative of the sensed patient physiological parameter;
activating, deactivating, or otherwise modulating the at least one electrode with the controller as a function of the sensor signal.

7. The method of claim 5, further comprising:
determining based on a first temporary period whether the patient is responding to the baroreflex therapy as generated by the external controller;
implanting an implantable baroreflex activation therapy pulse generator including a header adapted to connect with the connector in response to a determination that the patient is responding to the baroreflex therapy as generated by the external controller;
disconnecting the baroreflex activation device from the external controller; and
connecting the baroreflex activation device to the implantable baroreflex activation therapy pulse generator to provide baroreflex for a second chronic period.

8. The method of claim 5, further comprising:
disconnecting the lead from the baroreflex activation device after completion of a therapy period; and
removing the lead from the patient.

9. The method of claim 5, wherein the baroreflex activation device is implanted intravascularly.

10. The method of claim 5, wherein the baroreflex activation device is implanted extravascularly.

11. A baroreflex therapy system for providing temporary treatment to a patient, comprising:
an implantable baroreflex activation device having at least one electrode and including a lead with a first end coupled to the at least one electrode and a second end with a connector; and
an external controller, including a pulse generator adapted to deliver baroreflex therapy pulses to the implantable baroreflex activation device via a cable having one end adapted to releasably couple with the connector of the lead of the implantable baroreflex activation device, the controller adapted to activate, deactivate or otherwise modulate the implantable baroreflex activation device based on a programmed parameter and not in response to any sensed condition of the patient.

12. A method of treating a patient, comprising:
providing a baroreflex activation device having at least one electrode;
providing an external controller, including a pulse generator and a junction;
providing a lead coupled to the baroreflex activation device, the lead including a distal end configured to be coupled to the junction;
providing instructions to treat the patient, including:
implanting the baroreflex activation device proximate a baroreceptor in a vascular wall;
positioning the distal end of the lead through the skin of the patient such that the distal end of the lead is outside the body of the patient;
coupling the lead to the controller; and
activating, deactivating, or otherwise modulating the at least one electrode with the controller based on a programmed parameter and not in response to any sensed condition of the patient, to cause a baroreflex in the patient.

13. A therapy system, comprising:
a baroreflex activation device having at least one electrode, the baroreflex activation device configured to be proximate the exterior of the skin of a patient;
an external controller, including a pulse generator;
a sensor, configured to generate a sensor signal indicative of a patient physiological parameter other than blood pressure, the sensor coupled to the controller such that the controller activates, deactivates or otherwise modulates the at least one electrode as a function of the sensor signal.

14. A method of treating a patient, comprising:
providing a baroreflex activation device having at least one electrode;
providing an external controller, including a pulse generator;
providing a sensor coupled to the external controller;
positioning the baroreflex activation device proximate the exterior of the skin of a patient;
sensing a patient physiological parameter other than blood pressure with the sensor;
generating a sensor signal indicative of the sensed patient physiological parameter and transmitting the sensor signal to the external controller;
activating, deactivating, or otherwise modulating the at least one electrode with the external controller as a function of the sensor signal to cause a baroreflex in the patient.
